(19) United States
(12) Patent Application Publication
(10) Pub. No.: US 2006/0159631 A1
(43) Pub. Date: Jul. 20, 2006

(54) Compositions and methods for preventing dental stain

(75) Inventors: Robert Michael Buch, King of Prussia, PA (US); Alexander Couzis, King of Prussia, PA (US); Sean M. Wetterer, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION, Philadelphia, PA (US)

(21) Appl. No.: 10/548,890

(22) PCT Filed: Mar. 10, 2004
(86) PCT No.: PCT/US04/07265

§ 371 Date: Sep. 12, 2005

Related U.S. Application Data
(60) Provisional application No. 60/454,980, filed on Mar. 14, 2003.

(51) Int. Cl. A61K 8/55; A61K 8/73; A61K 8/46

(52) U.S. Cl. 424/49

(57) ABSTRACT

This invention relates to compounds and their use in oral hygiene compositions as anti-stain, anti-calculus, anti-bacterial, and anti-malodor agents. These agents are characterized by having two basic building blocks, one with the ability to strongly bind to the tooth surface by containing a functional group that allows the molecule to adsorb to the surface of teeth, the other by having a number of chemical moieties that are capable of binding strongly to water, preferably, to at least two water molecules.

### FIELD OF THE INVENTION

This invention relates to novel molecular agents and their use in oral hygiene compositions as anti-stain, anti-calculus, anti-bacterial, and anti-malodor agents.

### BACKGROUND OF THE INVENTION

Several factors contribute to tooth discoloration, but the three main factors are believed to be: (i) formation of plaque and tartar matrices on the tooth surface which then entraps stains; (ii) ingestion of certain drugs during gestational tooth formation; and (iii) discoloration due to oral cavity traumatization following which blood break-down products seep into the mineralized area of the teeth during enamel formation. This invention is primarily concerned with the first factor of tooth discoloration, that is, the natural stain that accumulates on teeth.

The active compounds disclosed by the instant invention reduce and/or prevent extrinsic stain. Over time and repeated remineralization/demineralization cycles, extrinsic stain can become incorporated into the outer layers of the tooth, thereby defined as intrinsic stain. The active compounds disclosed by the instant invention are also effective in reducing and/or preventing this type of intrinsic stain, i.e., the intrinsic stain originates from the long-term presence of or “the maturation of” extrinsic stain on dental surfaces.

The adsorption or deposition of foreign materials onto dental surfaces can have several undesirable effects. In the simplest case, colored molecules can directly adsorb onto the surface of teeth to produce a local discoloration. The adherence of bacteria to dental surfaces and the subsequent development of bacterial colonies can also discolor teeth as well as promote the formation of plaque, tartar, and calculus deposits. The activity of these bacteria may also have implications for malodor, tooth decay, and the overall sanitary state of the oral cavity.

Traditionally, consumers have used dentifrices or mouthwashes to restore the condition of dental surfaces by the removal of surface entrapped agents. The effectiveness of these products can be visually determined by the level of whiteness or luster that is recovered after use or by other sensory cues such as fresh breath and clean mouth feel. While these products generally contain cleaning and/or abrasive agents, some enamel deposits may resist complete removal under normal use conditions. Designed for frequent and repeated application, these preparations may not have been formulated with the quantity or type of agent required to completely remove extensive or tenacious deposits.

For the occasional removal of difficult adsorbates, there are various approaches currently in general use. One approach is the physical abrading of the contaminated enamel with formulations that contain polishing agents—harsher abrasives than those used in typical dentifrice preparations. Contaminant removal, in particular stain removal, can also be achieved by using oxidizing agents. Compositions containing urea peroxide, hydrogen peroxide, or calcium peroxide are commonly used to react with the components of the stain in order to form materials that are either colorless or more readily removed.

An alternative approach is to identify agents that will prevent the adsorption of undesirable contaminants, thus characterized as stain prevention agents. Dentifrice preparations have been disclosed previously which contain a polymer resin (e.g., polyvinylpyrrlidone) which is chosen to act as a physical barrier to the adsorption of the molecules responsible for the appearance of stain. Examples of these formulations are disclosed in U.S. Pat. No. 5,538,714, issued Jul. 23, 1996, to Pink et al., describing a method of reducing the adherence of oral bacteria to tooth enamel comprising applying to the tooth enamel a composition containing an anti-adherence effective amount of polyvinylpyrrolidone (“PVP”), GB Patent 739,936, published Nov. 2, 1955, disclosing compositions containing both chlorophyll and water-soluble PVP for inhibiting or preventing the formation of greenish stains associated with chlorophyll on certain absorbent materials such as cellulose, animal and synthetic fibers, and GB Patent 741,315, published Nov. 30, 1955, disclosing dentifrices containing PVP as a stain remover, particularly for tar-like stains. In each case, it is proposed that the presence of a large, relatively unreactive polymeric molecule protects dental surfaces.

In a continuous effort to improve oral hygiene and aesthetic perceptions, the inventors have identified compounds that prevent the adsorption of undesirable contaminants on the surfaces of natural teeth or dental prostheses.

### SUMMARY OF THE INVENTION

This invention relates to a dentally acceptable composition for preventing the accumulation of surface adsorbates on dental surfaces, including natural teeth and prosthetics, wherein the composition comprises at least one agent having a first moiety that enhances the persistence of the agent on dental surfaces and a second moiety capable of binding to at least two water molecules, in a suitable carrier.

In addition, this invention relates to a dentally acceptable composition for preventing the accumulation of surface adsorbates on dental surfaces, including natural teeth and prosthetics, wherein the composition comprises a combination of pegylated lauryl phosphate ether (DLP-0) and an agent selected from water soluble cellulose derivatives and water soluble polyphosphate salts, in a suitable carrier.

Further, this invention relates to methods for preventing the accumulation or reducing the quantity of surface deposited stains, bacteria, plaque, tarter, and calculus, on dental surfaces comprising contacting the surface of the tooth or prosthetic with a composition comprising at least one agent having a first moiety that enhances the persistence of the agent on dental surfaces and a second moiety capable of binding to at least two water molecules, in a suitable carrier.

Yet further, this invention relates to a method for preventing oral malodor comprising contacting the surface of the tooth or prosthetic with a composition comprising at least one agent having a first moiety that enhances the persistence of the agent on dental surfaces and a second moiety capable of binding to at least two water molecules, in a suitable carrier.

Still further, this invention relates to a method for preventing the accumulation or reducing the quantity of surface deposited stains, bacteria, plaque, tarter, and calculus, on dental surfaces comprising contacting the surface of the tooth or prosthetic with a composition comprising a combination pegulated lauryl phosphate ether (DLP-10) and a tooth whitening agent selected from water soluble cellulose derivatives and water soluble polyphosphate salts.

### DETAILED DESCRIPTION OF THE INVENTION

The following words are intended to be given the same meaning here as would be accorded to them in their contemporary usage in the oral and dental care arts. More specific usage for the invention herein is described below.

The term “anti-malodor” as used herein means capable of reducing the rate of formation of unpleasant odors in the oral cavity. It will be understood that dental/oral surfaces treated according to the invention should be less likely to accumulate food particles and bacteria which contribute to the formation of volatile odor compounds.

The term “anti-microbial” as used herein means capable of preventing, removing or reducing the presence of microbes, including bacteria. It will be understood that dental/oral surfaces treated according to the invention should resist the accumulation of bacteria thereby reducing the likelihood of colony formation.

The terms “calculus” and “tartar” as used herein interchangeably, mean, partially mineralized deposits on dental surfaces formed by the maturation of plaque.

The terms “anti-calculus” and “anti-tartar” as used herein interchangeably mean capable of preventing, removing or reducing the presence of calculus or tartar. It will be understood that the dental/oral surfaces treated according to the invention should have reduced formation of calculus/tartar, since formation is highly dependent on the presence of the bacteria which should be less likely to adsorb to treated surfaces.

The terms “dentally acceptable composition” or “orally acceptable composition” as used herein interchangeably mean a suitable vehicle that can be used to apply the present composition to the oral mucosa.

The terms “stain” or “staining” are used herein interchangeably with discoloration and generally mean that the surface of the enamel (or prostheses) has taken on some unwanted or unnatural coloration distinct from the color of the underlying enamel.

The terms “anti-stain” and “stain prevention” as used herein interchangeably, mean capable of inhibiting, removing or reducing the build-up of factors associated with stain on the tooth surface, including, but not limited to bacteria, plaque or calculus. It will be understood that the dental/oral surfaces treated according to the invention should resist the adsorption of potentially staining molecules.

Without being limited to a particular mechanism of action, we propose the following theory of stain prevention for this invention. It is believed that the instant active compounds or agents are capable of manipulating the surface energy of the tooth. This is accomplished by (i) reducing the adhesive strength between the stain components and the tooth enamel surface leading to displacement of the stain components from the tooth surface and (ii) by generating a tooth surface exhibiting low interfacial tension with saliva or water and exhibiting a high interfacial tension with the stain components preventing their deposition on the tooth surface.

It has been found that the instant agents reduce the interfacial tension between the dental surfaces, for example, tooth enamel, and the surrounding environment, for example, saliva, thereby leading to strong stain removal and stain prevention action. On such treated surfaces, contaminants are readily displaced by water, saliva, or other fluids present in the mouth thus maintaining the condition of dental surface. Such benefit is not typically provided by the application of polymer resins as described in the literature.

The inventive agents consist of two basic building blocks. One has the ability to strongly bind to the tooth surface by containing a functional group that allows the molecule to adsorb to the surface of teeth, presumably via chemical or electrostatic interactions. Such groups include, but are not limited to, sulfonates and sulfates, sulfosuccinates, taurates, phosphoric and polyphosphoric acid esters, sarcosinates. The other building block must contain a number of chemical moieties that are capable of binding strongly to water, preferably, to at least two water molecules. Such groups are include but are not limited to ethoxylates, polyethoxylates, and cellulose. When adsorbed on the surface, these molecules are thought to reduce the interfacial tension of the surface. Stain molecules and other contaminants which attempt to adsorb to a treated surface will be unable to bind strongly and can thus, be displaced by water.

### EXAMPLES

The following examples further describe and demonstrate preferred embodiments within the scope of the present invention. These examples are provided by way of illustration and are not intended to limit the scope of the invention. All proportions and amounts herein and elsewhere in this specification are by weight percent unless otherwise indicated.

### Example 1

Na-POE(10), a Lauryl Ether Phosphate (manufactured by Nikkol Company of Japan, and distributed in the U.S. by Barnett of New Jersey), of the general structure shown in FIG. 1, 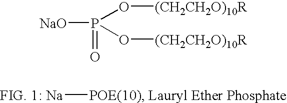
was dissolved in an aqueous solution to a concentration of 1% by weight. Hydroxyapatite (“HAP”) disks (manufactured by Clarkson Chromatography) with a diameter of 0.38″ were immersed in the above solution for 30 minutes. Before immersion, the water contact angle on the HAP disks was greater than 50 degrees. After treatment the water contact angle dropped to values below 5 degrees, and water droplets exhibited fast spreading behavior. Treated disks as well as bovine teeth treated in the same way showed no detectable staining after immersion in coffee for as long as 24 hours.

### Example 2

Sodium Carboxymethyl Cellulose, AQUALON 7MF (manufactured by Hercules Co.) as shown in FIG. 2, 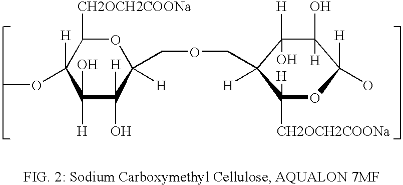
was dissolved in an aqueous solution to a concentration of 0.50% by weight. HAP disks with a diameter of 0.38″ were immersed in the above solution for 30 minutes. Before immersion the water contact angle on the HAP disks was greater than 50 degrees. After treatment the water contact angle dropped to values below 10 degrees, and water droplets exhibited fast spreading behavior. Treated disks as well as bovine teeth treated in the same way showed no detectable staining after immersion in coffee for as long as 24 hours.

### Example 3

Combinations of chemical agents were shown to produce favorable synergies for stain prevention. In a series of experiments, HAP discs were first treated with saliva to generate an artificial pellicle. (The presence of a pellicle was observed to greatly increase the likelihood of staining and was applied in order to discriminate between the various treatment cycles.) Following a thorough rinse, the pellicle-treated discs were then immersed in a dilute aqueous solution containing the active compound(s) under investigation. Following a second rinse, the active-treated discs were immersed in a concentrated solution of coffee for a period of 24 hours in order to simulate a dietary stain challenge. After rinsing to remove excess coffee solution, the discs were dried and rated for stain by visual inspection. Using a subjective index of 1 to 10 (1 representing greatest stain), stain intensity is tabluated below.

| Active Compound (w/w) | Stain Intensity |
|---|---|
| 2% DLP-10 | 3.5 |
| 5% DLP-10 | 4.0 |
| 10% STP | 5.5 |
| 5% SHMP | 6.5 |
| 2% DLP-10 + 10% STP | 7.5 |
| 2% DLP-10 + 5% SHMP | 8.5 |
| None | 2.0 |

Although all treatments permitted the development of some level of stain in this set of experiments, the combinations of DLP-10 and either STP or SHMP significantly outperformed any individual active compound.

The instant compositions may be presented in any of the conventional formulations such as dentifrices (including toothpastes), gels, mouthwashes or formulations that are chewed or sucked by the user such as a lozenge or a chewing gum. The instant compositions may also be presented in dissolvable and non-dissolvable films.

These formulations will be presented so that they are safe for use in the oral cavity and will not have a deleterious effect if accidentally swallowed. The oral care art has developed a substantial body of formulation types and has identified and tested a large list of ingredients useful in these formulations. Confecting or manufacturing these preparations, and their safe packaging and storage is also well documented.

In addition to the active ingredients, formulations for toothpastes, liquid pastes, gels and toothpowders suitable for this invention will contain the usual carriers, binders, surfactants, humectants, coloring agents, pigments, antiplaque agents, anti-bacterial agents, bioadhesive-type agents, abrasives, anticaries agents, flavorings, sweeteners, bulking agents, and the like.

Suitable abrasives for use in the present invention include precipitated silica, plastics particles, alumina, calcium carbonate, insoluble phosphates (e.g., zinc orthophosphate, dicalcium phosphate) and insoluble pyrophosphates. Pyrogenic silicas are not claimed as a useful silica for the instant invention. Silica is an especially preferred abrasive for use herein.

The patent and scientific literature is replete with examples of such abrasives. U.S. Pat. No. 4,822,599 listing a series of dentifrice abrasives, also references commercial sources and methods for their preparation.

Silica abrasives are well known and commercially available, generally having an average particle size ranging between about 0.1 to about 30 microns, such as from about 5 to about 15 microns. Silica dental abrasives useful in the present invention include those marketed by the J.M. Huber Corporation under the trade name Zeofree™ (Zeodent 113) and the silica xerogels marketed by the W.R. Grace and Company, Davison Chemical Division under the trade name ‘Syloid’. U.S. Pat. No. 3,358,230 and U.S. Pat. No. 3,862,307 describe silica dental abrasives useful in the toothpaste compositions according to the present invention. The silica abrasive may also be a naturally occurring amorphous silica such as diatomaceous earth. Suitable forms of diatomaceous earth are those marketed under the trademark ‘Celite’ by Johns-Manville Products Corporation, for instance ‘Celite Superfine Superfloss’.

The selected abrasive should be compatible with the actives. In addition, as with any other paste, gel or powder, the selection of an abrasive can be influenced by the consequence of combining a particular abrasive with another additive. For example, if fluoride ions and calcium pyrophosphate ions are to be included in these preparations the pyrophosphate should be converted from its γ-phase to its β-phase by heating the γ-phase to 700°-900° C. as per the teachings of U.S. Pat. No. 3,112,247. Also certain quaternary ammonium-based antibacterial agents may not be compatible with some silica abrasives.

Plastics dental abrasives are well known and are described in, for example, GB 939 230, GB 995 351, GB 1 055 784, and U.S. Pat. No. 3,151,027.

Alumina abrasives are well known and commercially available. Preferably the alumina abrasive may be treated with a solution of a surface-treating agent which may be an alkali metal silicate, hydrogen peroxide, an acid or an organophosphorus compound, of which an alkali metal silicate is especially preferred, as described in U.S. Pat. No. 4,781,982 (to Aluminum Company of America).

A calcium carbonate abrasive is preferably used in conjunction with an ionic agent to suppress the formation of free calcium ions, such as an alkali metal carbonate or bicarbonate, or mixture thereof, as described in EP 0 092 929 (to Beecham Group p.l.c.).

Abrasive concentrations can cover a very broad range. Preparations are described with abrasive ranging in concentration from 5 to 80% by weight depending on the abrasive. A secondary concentration range is that of 10 to 50% depending on the abrasive selected. Herein the preferred abrasive, silica, is employed in amounts between 5 and 30% by weight.

A source of fluoride ion may be included in the instant composition. Fluoride ion sources are numerous. For example, see U.S. Pat. No. 3,535,421 listing numerous salts useful in the dental arts. While any one of these sources could be used, sodium fluoride, sodium monofluorophosphate and stannous fluoride are considered the preferred ion sources in most dentifrices.

Fluoride ions are routinely added into dentifrices in an amount sufficient to provide up to 3500 ppm, preferably 1100 ppm of the fluoride ion. Where a preparation is formulated such that the fluoride ion is confined to one component of the preparation, but is mixed with the other components at the time of use, the fluoride ion source should be adjusted upward in an amount sufficient to provide a concentration of up to about 3500 ppm, but preferably 1100 ppm, in the product as used.

Suitably, in compositions of the present invention, the orally acceptable vehicle may comprise other components such as, flavorings, coloring agents, sweeteners, humectants, thickening agents, binders and surfactants.

Binders and thickening agents can be added to assure physical integrity in pastes, gels, films and liquid pastes. Preferred thickening and binding agents include for example natural and synthetic gums such as xanthan and acacia gums, carageenans, alginates, cellulose ethers and esters such as carboxy methyl cellulose, polyoxyalkyl polymers such as the Pluronics polymers, PVP materials, certain polymers exemplified by the carboxyvinyl polymers (Gantrez and the like), and silica. When the abrasive is silica, it is preferred to use a thickening silica as the thickening agent. A preferred thickening silica for use herein is Zeofree™ 153, which is a precipitated synthetic amorphous silica.

Binders are usually added in amounts ranging between 0.1 and 5.5% by weight.

Humectants are added to gels, films and pastes to prevent their drying out on exposure to air. In addition, they impart a “moist” feel to the mouth when brushing. Some humectants, e.g., sorbitol, are perceived as sweet. Examples of compounds useful as humectants in dentifrices are the polyhydric alcohols such as glycerin, sorbitol, propylene glycol and polyethylene glycols. A preferred humectant system consists of glycerin, sorbitol (usually 70% sorbitol/water) and polyethylene glycol, which is present in an amount of about 25-45%, preferably 37-40% of the total composition. In pastes and gels one to three humectants are usually used in amounts between about 10 and 80%. Preferably the humectants are used in amounts between about 20 and 50% of the total composition.

In addition, the orally acceptable vehicle may optionally comprise surfactants, sweetening agents, flavoring agents, anticaries agents (in addition to a fluoride ion source provided as a phosphatase enzyme inhibitor), anti-plaque agents, anti-bacterial agents such as triclosan or cetyl pyridinium chloride, tooth desensitizing agents, coloring agents and pigments.

Surfactants normally are added to dentifrices to assist with removing debris. Useful surfactants include the water-soluble salts of alkyl sulfates having from 10 to 18 carbon atoms in the alkyl moiety, such as sodium lauryl sulfate, but other anionic surfactants may also be used, e.g., non-ionic, zwitter-ionic, cationic and amphoteric surfactants. These compounds, and those which are most useful in the dental arts, are well documented in the literature. Reference is made to U.S. Pat. No. 4,822,599 for a detailed listing of useful surfactants. Surfactants are available through a number of commercial manufacturers or can be made by well documented processes.

Surfactants are normally used in amounts between about 0.5 and 5% by weight in pastes and gels but may be used at higher concentrations in some dental powders. Surfactants can also be used as gelling agents.

Taste is provided by adding a small amount of a flavoring agent to the composition. Numerous minty flavored agents are available for use in dentifrices. It is well known in the art how to select a flavoring agent which will be acceptable to consumers. Flavoring agents are routinely used at levels of between about 0.1 to 5% by weight.

Dyes, lakes and titanium dioxide are routinely used in the dentifrice arts for imparting color to the composition. When titanium dioxide is the coloring agent, a white paste or powder is obtained. These materials are widely available and are well known to the dental artisan. Coloring agents are usually present in concentrations ranging between 0.0001 and 5%.

Sweeteners are routinely added to increase consumer acceptability. Artificial sweeteners are used today to avoid the cariogenic potential of most sugars and other sweetening agents. Examples of non-cariogenic sweeteners now in routine use are saccharin, aspartame, D-tryptophan, dihydrochalcones, cyclamates, xylitol and acesulfame. Sweeteners comprise about 0.1 to 5% by weight of the formulation.

The active(s) can be formulated as a mouthwash or mouth rinse as well. A mouth wash or rinse will contain up to 95% water, up to 30% alcohol, flavor, polyhydric alcohols, anti-caries agents, plaque removing agents, sweeteners, dyes and lakes, and a preservative in some instances, and sufficient water to make volume. The active could also be incorporated into currently existing formulations such as Cepacol (Lakeside Pharmaceuticals), Plax, (Pfizer), Scope (Procter & Gamble), and the like.

A soaking and cleaning solution for dental pieces can also be prepared with the combination of active ingredients. It is contemplated that such preparations would contain water, a surfactant, an effervescing agent, and other optional ingredients. Dental prostheses would be removed and placed in a solution containing the tripolyphosphate salt and pyrophosphate salt, and soaked for several hours, then either brushed with a recommended dentifrice or simply rinsed and reinserted into the mouth.

When the preferred aqueous orally acceptable dental vehicle is employed, a toothpaste composition of the present invention suitably contains from about 10 to about 80% humectant, from about 0.25 to about 5% detergent, from 0 to about 5% sweeteners and flavoring agents together with water and an effective amount of binding and thickening agents, such as from about 0.1% to about 12%, to provide the toothpaste of the invention with the desired stability and flow characteristics.

Conventional manufacturing techniques are employed to prepare a toothpaste with the inventive active combination. Toothpaste compositions of the present invention may also be prepared in the form of a clear gel or a paste of a uniform color or in the form of a striped toothpaste. A suitable apparatus for filling toothpaste tubes with striped toothpaste is described in GB 962 757. In accordance with this patent, toothpastes of different colors are fed through separate tubes of a bundle of tubes that is inserted into a toothpaste container and gradually moved relative to the container as the container is filled.

The toothpaste of the invention is used in a conventional manner by applying the toothpaste to the teeth. Most dentists and researchers recommend brushing one's teeth for at least three minutes per brushing to achieve maximum results, although compliance with this standard is not universal. A similar standard is recommended for the instant pastes and gels, although it is expected that non-compliance will still provide the desired results with regular use, i.e., daily use.

All publications, including, but not limited to, patents and patent applications cited in this specification, are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

What is claimed is:

1. A dentally acceptable composition for preventing the accumulation of surface adsorbates on dental surfaces wherein the composition comprises at least one agent having a first moiety that enhances the persistence of the agent on dental surfaces and a second moiety capable of binding to at least two water molecules, in a suitable carrier.

2. The composition as in claim 1, wherein the first moiety is selected from sulfonates and sulfates, sulfosuccinates, taurates, phosphoric and polyphosphoric acid esters, sarcosinates.

3. The composition as claimed in claim 1, wherein the second moiety is selected from ethoxylates, polyethoxylates, and cellulose.

4. The composition as claimed in claim 1, wherein the agent is present in an amount between about 0.001% percent by weight and 50% percent by weight.

5. A method for preventing the accumulation or reducing the quantity of surface deposited stains from dental surfaces which method comprises contacting the surface of the tooth or dental prostheses with a composition according to claim 1.

6. A method for preventing the accumulation or reducing the quantity of bacteria on dental surfaces which method comprises contacting the surface of the tooth or dental prostheses with a composition according to claim 1.

7. A method for preventing the accumulation or reducing the quantity of plaque on dental surfaces which method comprises contacting the surface of the tooth or dental prostheses with a composition according to claim 1.

8. A method for preventing the accumulation or reducing the quantity of tartar on dental surfaces which method comprises contacting the surface of the tooth or dental prostheses with a composition according to claim 1.

9. A dentally acceptable composition for preventing the accumulation of surface adsorbates on dental surfaces wherein the composition comprises a combination of pegylated lauryl phosphate ether (DLP-10) and an agent selected from water soluble cellulose derivatives and water soluble polyphosphate salts, in a suitable carrier.

10 The composition as claimed in claim 9, wherein the agent is sodium carboxymethyl cellulose.

11. The composition as claimed in claim 9, wherein the agent is sodium tripolyphosphate.

12. The composition as claimed in claim 9, wherein the agent is sodium hexametaphosphate.
